(19) United States
(12) Patent Application Publication
(10) Pub. No.: US 2002/0151089 A1
(43) Pub. Date: Oct. 17, 2002

(54) Separating components of biological samples

(76) Inventors: William H. Chapman JR., San Leandro, CA (US); Leonard Klevan, Orinda, CA (US)

Correspondence Address: TIMOTHY A. PORTER, Fish & Richardson P.C., Suite 500, 500 Arguello Street, Redwood City, CA, 94063, US

(21) Appl. No.: 10/072,295

(22) Filed: Feb. 5, 2002

Related U.S. Application Data
(63) Continuation-in-part of application No. 09/802,381, filed on Apr. 16, 2001.
(60) Provisional application No. 60/154,148, filed on Sep. 15, 1999.

(51) Int. Cl. G01N 33/543

(52) U.S. Cl. 436/523

(57) ABSTRACT

Methods, compositions and systems for processing biological samples include separation reagents featuring a microparticle and a receptor for a ligand on a target species in the biological sample. The biological sample is reacted with the separation reagent to capture the target species. A covalent bond is formed between the target species and the separation reagent to form an adduct. The adduct is separated from the biological sample, and a component of the target species is separated from the target species.

### CROSS-REFERENCE TO RELATED APPLICATIONS

This application is a continuation-in-part of application Ser. No. 09/802,381, filed Sep. 15, 2000, which claims the benefit of Provisional Application No. 60/154,148, filed Sep. 15, 1999, both of which are incorporated by reference herein.

### BACKGROUND OF THE INVENTION

The present invention relates to methods, compositions and systems for isolating materials from biological samples. Multiplexed short tandem repeat (“STR”) analysis of human DNA has been found to be a very useful technique for the identification of persons for law enforcement purposes. In order for the data generated to be accepted in a court of law, the STR method requires the use of DNA from a single individual that has been isolated, purified and quantified using a highly reproducible protocol. When applied to the analysis of sexual assault evidence, difficulties are encountered because the samples which are collected often contain cellular material from both the victim and the suspect. Extraction of the total DNA from the cellular mixture yields DNA from both persons in an unknown ratio, often with more of the victims DNA than the suspect's. Current protocols call for the separation of the male (sperm) and female (e.g. epithelial) components before the DNA isolation step by using a single differentiating feature of these cell types: epithelial cells tend to lysis more quickly with detergents than do sperm cell (under non-reducing conditions). There is no a priori reason to expect these protocols to yield a complete separation of the male and female fractions. If the sample contains far more of the victims DNA then the suspects, which is often the case, then STR analysis often reveals only the victim's genotype, leaving the suspect unidentified.

Much effort has been invested in the generation of highly selective antibodies for binding to specific biological targets, such as human sperm. Hybridoma cell lines which produce antibodies which are found to be useful for in vitro assays (i.e. ELISA) and pharmaceutical applications (e.g. birth control) are commercially available (e.g. ATCC HB-9762, HB-255 and HB-10039).

Photoaffinity labeling has become a popular technique for studying the binding interactions between biomolecules which accompany most biological events. According to this technique, one biomolecule expected to be involved in a binding event is decorated with a chemical group which will form covalent bonds to a second involved biomolecule during or after light activation.

One popular photoaffinity labeling technique uses the arylazide group for the light activated attachment step. When light activated, arylazides lose diatomic nitrogen producing the reactive nitrene intermediate. Nitrenes are known to form covalent bonds to neighboring molecules by addition to unsaturated linkages or insertion into single covalent bonds (C—H or C—C). Thus, attachment of the arylazide group to one molecule allows it to be covalently coupled to a second molecule. This property of the arylazide group has been applied in the area of bioconjugation (e.g. biotin labeling) and protein crosslinking, as described in B. Lacey & W. N. Grant, Anal. Biochem. (1987) Vol. 163, p. 151 and U. C. Krieg, et al., Proc. Natl. Acad. Sci. USA (1986) Vol. 83, p. 8604.

Active esters of photoaffinity labels such as arylazides are commercially available and protocols for their use in decorating proteins are well developed, as described in D. A. Geselowitz & R. D. Neumann, Bioconjugate Chem. (1995) Vol. 6, p. 502 and A. C. Forster, et al., Nucleic Acids Research (1985) Vol. 13, p. 745. NHS activated esters, for example, will react with primary amino groups of proteins (e.g. lysine residues) producing stable amide bonds. Photoactivation of the decorated protein, after binding to a receptor, will produce a covalent adduct between the photoaffinity label and the receptor.

The automated isolation of DNA for PCR amplification is a topic of current interest. The development of magnetic bead methods for DNA isolation is seen as being a generally useful activity, increasing the throughput and reproducibility of the PCR method as a whole. Many new technologies of this type have appeared recently which have not been tested for STR amplification. For example, Dynal Corporation has introduced a magnetic bead protocol which has become quite popular in molecular biology research and is finding applications in the clinical laboratory. Hardware for the automation of the Dynal method is commercially available (Biomek 2000, Beckman Coulter, Inc., Fullerton, Calif.). Automated assays employing magnetic beads, such as the Isolex 300i system for CD34 cell isolation available from Nexell Therapeutics, Inc., of Irvine, Calif., have reported and are commercially available.

### SUMMARY OF THE INVENTION

In general, in one aspect, the invention features methods for processing biological samples. The methods include providing a separation reagent that includes a microparticle and a receptor for a ligand on a target species in the biological sample; reacting the biological sample with the separation reagent to capture the target species; creating a covalent bond between the target species and the separation reagent to form an adduct; separating the adduct from the biological sample; and separating a component of the target species from the target species.

Particular embodiments can include one or more of the following features. The covalent bond can be formed by activating a photoaffinity label coupled to the separation reagent. The photoaffinity label can be coupled to the receptor. The photoaffinity label can be coupled to the receptor at an N-terminus. Separating the adduct can include, for example, magnetically capturing the microparticle, or capturing the microparticle by filtration or centrifugation. The receptor can include at least one binding protein, such as an antibody. The biological sample can be a forensic sample and the target species can be a sperm cell. The separated component of the target species can include a DNA. The microparticle can have a diameter in the range of from about 1 millimeter to 200 nanometers, from about 1 millimeter to about 500 nanometers, or from about 1 millimeter to about 1 micrometer.

In general, in another aspect, the invention features separation reagents for separating components of biological samples. The separation reagents include a microparticle, a receptor coupled to the microparticle, and a photoaffinity label coupled to the receptor.

Particular embodiments can include one or more of the following features. The photoaffinity label can be coupled to the receptor at an N-terminus, and can be an arylazide, such as a nitroarylazide. The photoaffinity label can also be sulfosuccinimidylperfluoroazidobenzamido-ethyl-1,3′-dithiopropionate, sulfosuccinimidyl-2-[m-azido-onitrobenzamido]ethyl-1,3′-dithiopropionate, N-succinimidyl-4-azidophenyl-1,3′-dithiopropionate or sulfosuccinimidyl 2-[7-azido-4-methyl-coumarin-3-acetamido]ethyl-1,3′-dithiopropionate. The microparticle can include a magnetic bead. The receptor can include at least one binding protein, such as an antibody. The microparticle can have a diameter in the range of from about 1 millimeter to 200 nanometers, from about 1 millimeter to about 500 nanometers, or from about 1 millimeter to about 1 micrometer.

In general, in a third aspect, the invention features apparatus for separating components of a biological sample. The apparatus includes a first chamber for receiving a biological sample; a first capture means proximate to the first chamber for capturing a separation reagent; a second chamber in fluidic communication with the first chamber; and a second capture means proximate to the second chamber for capturing the separation reagent.

Advantages that can be seen in implementations of the invention include one or more of the following. Capturing the target species with a selective receptor provides a high degree of selectivity for the target species in the biological sample. The permanent attachment of the target species and separation reagent using a photoaffinity label allows for the complete separation of the target species from the biological sample. The use of microparticles provides a large surface area for the permanent attachment of receptors, enabling the efficient capture of a large percentage, or all, of the target species molecules from the biological sample. The use of magnetic beads for sample separation and DNA isolation avoids the need for centrifugation steps, thereby enabling the full automation of sample processing and yielding highly reproducible results.

The details of one or more embodiments of the invention are set forth in the accompanying drawings and the description below. Other features and advantages of the invention will become apparent from the description, the drawings, and the claims.

### BRIEF DESCRIPTION OF THE DRAWINGS

FIG. 1 is a flowchart illustrating a method of isolating a target species from a biological sample.

FIG. 2 illustrates the synthesis of a photoactivatable separation reagent according to the invention.

FIG. 3 illustrates the use of a photoactivatable separation reagent to isolate DNA from a target species in a biological sample.

FIG. 4 is a schematic diagram of an apparatus for isolating a target species from a biological sample according to the invention.

FIG. 5 is a chromatogram (absorbance at 280 nm) showing the elution of a reduced antibody and excess MEA from an acrylamide column.

FIG. 6 is a chromatogram (absorbance at 412 nm) showing the elution of a reduced antibody and excess MEA from an acrylamide column after treatment of a portion of each fraction with Ellman's reagent.

Like reference numbers and designations in the various drawings indicate like elements.

### DETAILED DESCRIPTION

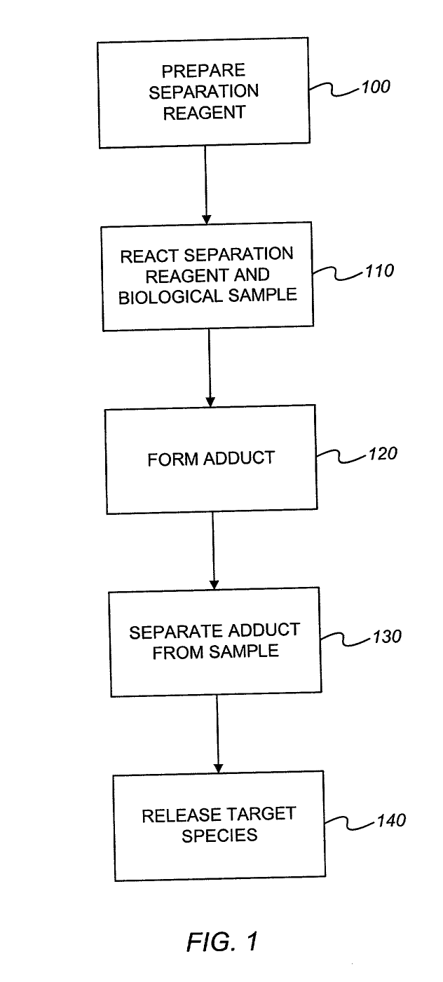

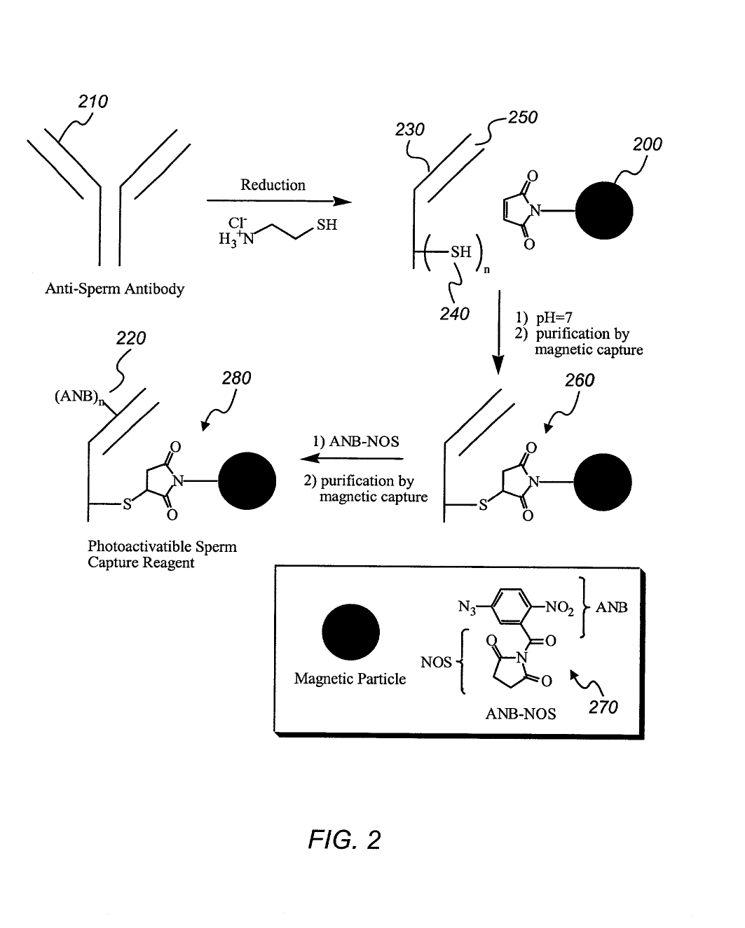

The invention provides methods, compositions and systems for processing biological samples using microparticulate separation reagents to capture a target species from a biological sample. As shown in FIG. 1, the methods begin with the preparation of a separation reagent (step 100). The separation reagent is formed by binding a receptor for example, a binding protein having affinity for a target species in the biological sample to a microparticle, such as a coated magnetic bead, as will be described in more detail in connection with FIG. 2 below. Microparticles for use in the invention generally have dimensions of from about 1 millimeter to about 1 nanometer, and may be fabricated from a wide variety of materials, including latex polystyrene, colloidal metals or other appropriate substances using known techniques. In some implementations, the microparticles have diameters in the range of from about 1 millimeter to 200 nanometers. In other implementations, the microparticle diameter is in the range of from about 1 millimeter to about 500 nanometers. In still other implementations, the microparticle diameter is in the range of from about 1 millimeter to about 1 micrometer.

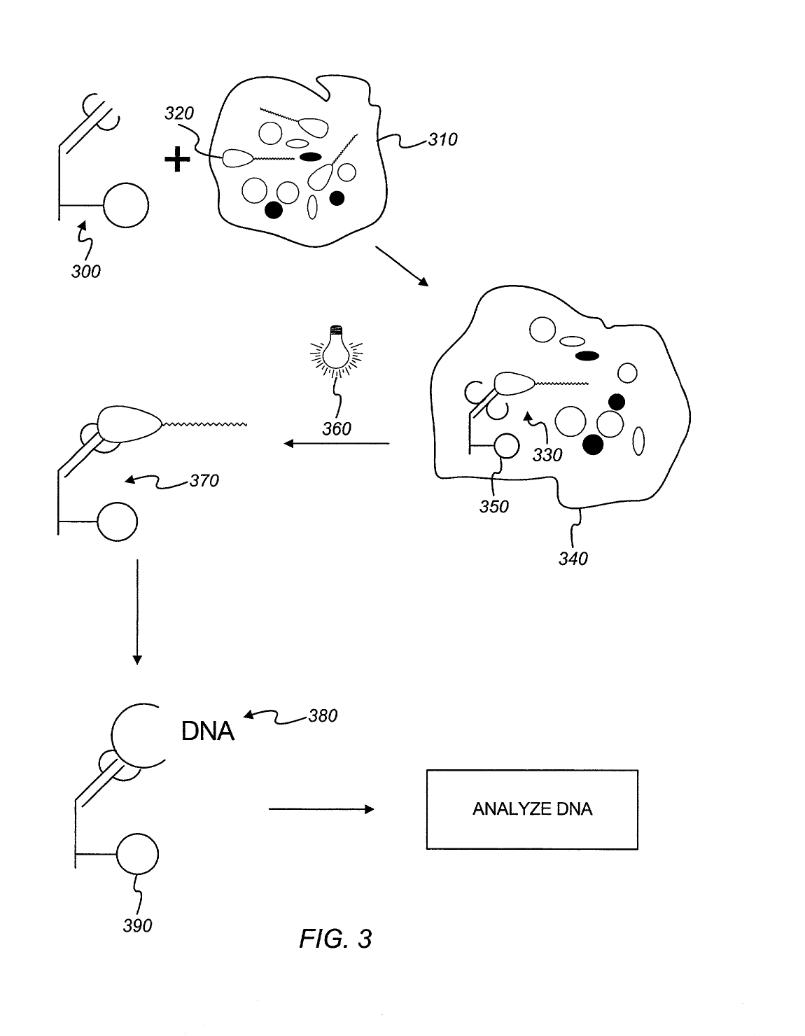

The prepared separation reagent is reacted with a biological sample known or suspected to contain the target species (step 110), and an adduct of the separation reagent and the target species is formed (step 120), as will be described in more detail in connection with FIG. 3 below. This adduct is separated from the biological sample (step 130), and the target species is released for further analysis free of the biological sample (step 140).

Referring to FIG. 2, in one embodiment photoactivatable separation reagents for specific target species are prepared by decorating microparticles 200 with target-specific receptors 210 and attaching a chemical species 220 to the receptors that will form covalent bonds between the receptor and the target species after photoactivation. In the described embodiment, the target species is human sperm and the receptor is a binding protein having an affinity for human sperm—for example, an anti-sperm antibody or mixture of anti-sperm antibodies such as those produced by cell lines ATCC HB-9762, HB-255 and HB-10039. Ideally, the receptor should (1) not be crossreactive with other cell types found in the biological sample (or be minimally crossreactive); (2) bind to the target species with high affinity; and (3) and in this embodiment, bind selectively to the head of the sperm cell. The selective binding to the sperm head is important because the head contains the DNA targeted for isolation and the tails of the sperms are often found missing in case samples.

Appropriate receptors can be identified by screening for affinity and the orientation of binding using known techniques—for example, microtiter plate based fluorescence assays and optical microscopy. Those skilled in the art will recognize that the principles of the invention can be applied advantageously to other targets and receptors, such as detection of pathogens in food, profiling organisms present in environmental samples, separating fetal blood cells from maternal blood cells for cytogenetic analysis, isolation of lymphocytes or other cells from whole blood, etc.

As shown in FIG. 2, selective reduction of the disulfides which connect the heavy chains of an antibody 210 (e.g. with 2-aminoethanethiol) produces antibody fragments 230 which carry reactive sulfhydryl groups 240 on a portion of the antibody fragment which is a distance from the Fab region 250 of the antibody binding site. Fragments 230 are attached to microparticles such as coated magnetic beads 200, such as those available from Dynal Corporation of Oslo, Norway, or Bangs Laboratories, Inc., of Fishers, Ind., by the reaction of sulfhydryl groups 240 with haloacetyl or maleimide groups on the microparticle surface, as described in K. Kato, et al., J. Immunology (1976) Vol. 116 (6), p. 1554. Optionally, fragments 230 are attached to microparticles 200 through an additional linker such as a secondary antibody. Because of the location of sulfhydryl groups 240, the chemical bonding of sulfhydryl groups 240 to the surface of the microparticles will thus place Fab region 250 a distance from the surface of the microparticle 200 such that it can bind to the target antigen with out interference.

After purification of adduct 260 by magnetic capture, filtration, centrifugation or other separation techniques, photoaffinity label 220 is introduced, e.g., by acylation with the NHS ester of a nitroarylazide, such as 5-azido-2-nitrobenzoyloxysuccinimide 270 (ANB-NOS, available from Pierce Chemicals) under known conditions. See U.C. Krieg, et al., Proc. Natl. Acad. Sci. USA (1986) Vol. 83, p. 8604. The nitroarylazide group has a red shifted absorbance spectrum relative to other known arylazides, making its photoactivation possible with visible light, which is advantageous for many biological samples of interest, such as those collected in sexual assault cases, which may contain UV absorbing impurities. Other photoactivatible cross-linking agents, such as sulfosuccinimidyl-perfluoroazidobenzamido-ethyl-1,3′-dithiopropionate (SFAD), sulfosuccinimidyl-2-[m-azido-o-nitrobenzamido]ethyl-1,3′-dithiopropionate (SAND), N-succinimidyl-4-azidophenyl-1,3′-dithiopropionate (SADP) or sulfosuccinimidyl 2-[7-azido-4-methyl-coumarin-3-acetamido]ethyl-1,3′-dithiopropionate (SAED) can be used. The selection of an appropriate cross-linking agent will be based on the particular application. Preferably, the acylation reaction introduces the photoaffinity label such that reaction with the N-terminus of the antibody fragment will be favored. Without intending to be bound by theory, it is believed that the N-terminus of the antibody is adjacent to the variable region (binding site) and thus is expected to be near the cellular antigen after the antibody binds to the cell. Other chemistries can be used to attach the photoaffinity label to antibody. Separation reagent 280 is then isolated by magnetic capture, filtration, centrifugation or other separation techniques, and washed to remove impurities.

Referring to FIG. 3, the binding of an intact sperm cell to the modified antibody, after light activation, produces a permanent (covalent) antibody-sperm cell adduct 370. The capture of the microparticles, followed by rigorous washing will allow for the permanent separation of the sperm cells, and most importantly the DNA that they carry, from any other cells or cellular debris present in the sample.

Separation reagent 300 is added to a solution of the biological sample to be analyzed 310, which is prepared by suspending the sample cells, collected, for example, from a sexual assault victim, in a suitable buffer (for example, containing surfactants for disruption of epithelial cells and the like). Optionally, low energy sonication is used to ensure that all target cells are extracted into solution 310. Separation reagent 300 captures target cell 320, forming receptor-ligand complex 330. In one embodiment, complex 330 is separated from mixture 340 by capture of microparticles 350, by magnetic capture, filtration, centrifugation or other separation techniques, and is resuspended in fresh buffer. In another embodiment, mixture 340 is carried directly to the irradiation stage, described next.

The suspension containing complex 330 is irradiated with light from light source 360, producing covalent adduct 370 and permanently attaching target cell 320 to bead 350. Adduct 370 is isolated by magnetic capture, filtration, centrifugation or other separation techniques, and purified by repeated washing, optionally including low energy sonication, until all traces of foreign cellular material (e.g., cellular material from a victim) are removed.

Because adduct 370 (or optionally complex 330) can be separated from mixture 340 by magnetic capture, no centrifugation step is necessary to isolate the target cells from the biological sample. As a result, the isolation of target cells, and analysis of target DNA, can be completely automated, for example using known techniques and hardware such as the Biomek 2000 available from Beckman Coulter, Inc. This automation results in increased efficiency and reproducibility as compared to previously known methods.

Target cell DNA 380 is released from adduct 370, for example by chemical reduction (e.g. buffer containing 2-mercaptoethanol or dithiothreitol) and/or proteinase K digestion or other means, and the microparticles 390 are removed from the sample by magnetic capture, filtration, centrifugation or other separation techniques, if desired. Purified DNA 380 is then analyzed using known techniques, such as the prior art magnetic bead/PCR techniques described above.

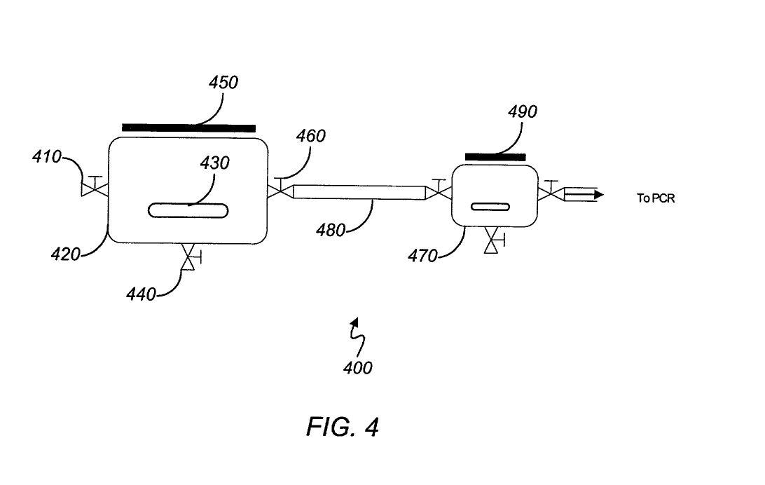

One embodiment of an apparatus 400 for applying these techniques to the processing of the forensics samples is illustrated in FIG. 4. A forensics sample suspected to contain a biological target species (e.g., sperm cells) is introduced through inlet 410 into a chamber 420 loaded with a separation reagent or reagents as described above. In this embodiment, the sample can be of varying volume (e.g., from 1 to 100 milliliters or more) and composition (e.g., clothing, upholstery, etc.), and inlet 410 and chamber 420 are configured accordingly. A buffer solution is introduced into chamber 420 and the target species is suspended in the solution, for example by mixing using stirrer 430. After the target species is captured by and attached to the separation reagent (to form an adduct as described above), the remaining solution is drained through outlet 440. Optionally, depending on the relative size of the separation reagent and outlet 440, the adduct can be captured prior to draining to prevent any loss of adduct (e.g., by magnetic interaction between electromagnet 450 and coated magnetic beads included in the separation reagent, by gravity, or other means as discussed above). The adduct can also be washed to remove all traces of contaminants by adding and draining additional solution through inlet 410 and outlet 430 respectively.

Optionally, the adduct is resuspended in clean buffer and valve 460 is opened to allow the mixture to flow to chamber 470 through tubing 480. As the mixture flows through chamber 470, the adduct is captured (e.g., by electromagnet 490). Chamber 470 is then sealed, and the target species is released from the captured adduct as described above. This results in a concentrated sample of the target species suitable for further analysis—for example, PCR amplification and analysis. In other embodiments, the second chamber 470 can be omitted, in which case the target species is released for further analysis after processing in the first chamber 420.

The following examples are provided by way of illustration and are not intended to limit the invention in any way.

### EXAMPLE 1

### Preparation of Magnetic Separation Reagent

In general, magnetic beads were decorated with an aryl azide labeled antibody using the chemical steps discussed above in the context of FIG. 2. An epoxide functionality on the magnetic beads was converted to a maleimide by reaction of the epoxide with a large excess of a diamine and acylation of the remaining amine with an activated ester that carries the maleimide group. The antibody to be attached was reduced with a thiol and conjugated to the beads by addition of a free sulfhydryl group to the maleimide. This antibody attachment protocol leaves the N-terminus of the protein available for acylation with the photoaffinity label. All chemicals used were purchased from the Aldrich Chemical Company (Milwaukee, Wis.) and were used without further purification unless otherwise stated. Antibodies were produced from cell lines purchased from the American Type Culture Collection (Manassas, Va.) by Rockland Immunochemicals Inc. (Gilbertsville, Pa.) and were stored in PBS buffer containing 0.01% (w/v) NaN₃ at 4° C.

Functionalization of Magnetic Beads with Amino Groups.

Epoxy activated magnetic beads (M450 Dynabeads from Dynal, A. S, Oslo, Norway) were transferred to a 1.5 mL tube (0.5 mL bead solution, 4×10⁸ beads/mL in water). 50 μL of (ethylenedioxy) bis(ethylamine) was added and the samples were mixed completely by vortexing. The 1.5 mL tubes were placed inside of a 50 mL falcon tube. Samples were heated to 50° C. with rapid (300 rpm) shaking (x-y rotating shaker) for 4 hours. The tubes were sonicated, vortexed and the beads were separated magnetically (Dynal MPC-S). 5 The clear colorless water/amine mixture was discarded and replaced with diglyme (800 μL). The brown pellet was resuspended by vortexing and was sonicated, vortexed (VSV) and magnetically separated. A second portion of diglyme was added, the samples were VSV and the tubes were agitated by inversion at 50 rpm for 1 hour. The process was repeated twice more for 15 minutes each with diglyme and twice with water. These beads were stored in 0.5 mL of water at 4° C.

Acylation of Functionalized Beads.

The functionalized beads were then decorated with the maleimide groups by acylation with SMPB (succinimidyl-4-[pmaleimidophenyl]butyrate, Pierce Chemicals, Rockford, Ill.). The beads (300 μL) were magnetically separated and resuspended in DMF (1 mL portions) three times and the maleimide NHS ester (SMPB) was added (50 μL, 50 mg/200 μL). The beads were stirred by inversion for three hours and were magnetically separated and washed with DMF (1 mL portions) four times. After washing was complete, the beads were resuspended in PBS buffer pH=7.00 (300 μL, 20 mM phosphate, 150 mM NaCl and 1 mM EDTA). The maleimide-decorated beads were reacted with the reduced antibodies immediately.

Decoration of Beads with Antibodies.

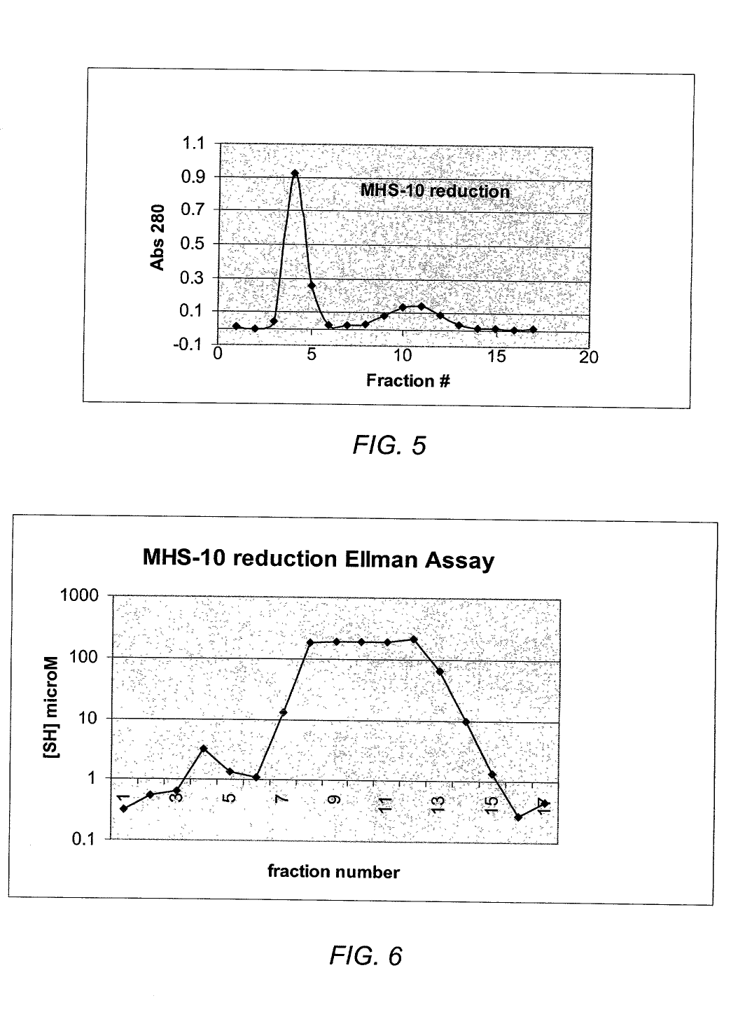

Antisperm antibodies (MHS-10 from ATCC HB-10039) were reduced with MEA (2-mercaptoethylamine) and the fragments were separated from excess MEA by column chromatography. MHS-10 (1 mL in PBS, pH=7.2, 1 mg/mL) was spiked with EDTA (1 mM, 2 μL 0.5 M solution) and added to MEA (14.5 mg, 128 mM) and incubated for 4 hours at 37° C. After this time, the sample was cooled to room temperature and loaded onto a polyacrylamide desalting column (Pierce Chemicals, D-salt 6000). The column was washed with 50 mL PBS buffer containing 1 mM EDTA (5 column volumes) before applying the sample. After loading the sample, 1 mL volume samples were taken for 20 mL of eluent. The contents of each fraction were determined by the absorbance at 280 nm and, after treatment with Ellman's reagent, 412 nm, as shown in FIGS. 5 and 6, respectively. Ellman's reagent (10 mM, 200 mg/50 mL) was prepared in 100 mM phosphate buffer (pH=8). 50 μL of the 10 mM Ellman solution was added to a 1 mL fraction (for a final concentration of 500 μM) and the sample was mixed and incubated at RT for 15 minutes. The concentration of the 4-nitrothiophenylate was determined at 412 nm. The concentration of thiols was calculated from the absorbance of the phenylate at 412 nm and a calibration plot generated with cystine standards. The concentration of protein was measured using a BSA calibration curve. The concentration of the antibody eluted is found to be 0.925 mg/mL or 6.167 μM (assuming 150,000 g/mol as the molecular weight of IgG₁). The Ellman assay showed that the thiol concentration in the same fraction is 31.1 μM. Unreduced MHS-10 antibody (1.5 mg/mL) was tested with Ellman's reagent and showed no measurable absorbance at 412 nm.

The reduced antibody (250 μL, 0.65 mg/mL) was added to the maleimide bead preparation (300 μL) and the mixture was mixed by inversion (60 rpm) for 2 hours at room temperature. The beads were magnetically separated, resuspended in 1 mL PBS buffer that contained 50 mM MEA and was mixed by inversion (60 rpm) for 1 hour. The thiol containing buffer was exchanged for fresh PBS and the washing process was continued overnight. The next day, the beads were washed with three more portions of PBS and were stored in 0.3 mL PBS at 4° C.

The presence and approximate concentration of the antibodies on the magnetic beads was determined by depletion of solutions of goat anti-mouse HRP in PBS containing 0.1% BSA. The concentration of the HRP was determined by reaction with luminol (Pierce SuperSignal ELISA Femto) and measurement of light intensity with a CCD imaging system. The number of antibodies bound per bead was between 10² and 10⁵.

Labeling of the Antibodies with Arylazides.

A solution of SFAD (sulfosuccinimidyl (perfluoroazido benzamido)-ethyl 1,3′ dithiopropionate, Pierce) was prepared in anhydrous DMF (100 mM). A portion (50 μL) of the antibody-conjugated beads was magnetically captured and washed three times with 10% DMF in phosphate buffer (pH=7.5, 50 mM). A portion of the stock solution of SFAD was diluted 10 fold in anhydrous DMF and 25 μL was added to the beads to give a final concentration of 10 μM. The samples were incubated at RT for 1 hour and the beads were captured. The beads were washed three times in the reaction buffer and were stored in 50 mM phosphate, pH=7.5 at 4° C. in the dark.

### EXAMPLE 2

Magnetic beads decorated with arylazide labeled antibodies as described above were tested (Professor Arthur Eisenberg, University of North Texas Health Science Center at Fort Worth) and found to be effective for the capture of human sperm from simulated forensics samples. Beads conjugated to the MHS-10 antibody that were modified with the aryl azide and beads that were not modified were tested in parallel and were found to be effective for the capture of sperm. However, the bead-sperm cell complexes formed with the beads that were not labeled with the aryl azide could be easily dissociated by vortexing, making the washing of the captured sperm-bead complex very difficult. The photoaffinity labeling event was found to strengthen the attachment of the sperm to the magnetic bead such that it was no longer disturbed by vortexing.

Antibody coated beads (1e6 beads) prepared according to Example 1 were added to human sperm samples (1e6 cells, donated by an unidentified 34 year old Caucasian donor) in PBS (0.5 mL) containing 0.1% BSA. BSA coated beads, prepared by the reaction of BSA with the epoxide magnetic beads, as described in technical literature from Dynal, were used as a control. The samples were incubated at 4° C. for 1 hour with inversion (circa 60 rpm). Samples were submerged in ice water and were photolysed for 5 minutes with a mercury vapor lamp (300 watts) while being gently agitated. After photolysis, the beads were captured with a stationary magnet (Dynal MPC-S) and the sperm cells remaining in the PBS were counted with a hemacytometer. The BSA coated beads were not found to deplete the sperm sample. The beads coated with the antibodies were found to deplete the sperm samples, removing consistently >80% of the sperm relative to the BSA control. Vortexing the samples before the magnetic capture was found to dissociate the sperm cells that were attached to the magnetic beads. After vortexing, the solution of beads that were modified with the photoaffinity label showed no increase in the concentration of the sperm cells. For comparison, the solution of beads that were coated with the antisperm antibody but were not modified with the aryl azide showed the presence of sperm cells similar to the BSA standard.

The invention has been described in terms of particular embodiments. Those skilled in the art will recognize that other embodiments are within the scope of the following claims.

What is claimed is:

1. A method of processing a biological sample, comprising:
providing a separation reagent comprising a microparticle and a receptor for a ligand on a target species in the biological sample;
reacting the biological sample with the separation reagent to capture the target species;
creating a covalent bond between the target species and the separation reagent to form an adduct;
separating the adduct from the biological sample; and
separating a component of the target species from the target species.

2. The method of claim 1, wherein:
the covalent bond is formed by activating a photoaffinity label coupled to the separation reagent.

3. The method of claim 2, wherein:
the photoaffinity label is coupled to the receptor.

4. The method of claim 3, wherein:
the photoaffinity label is coupled to the receptor at an N-terminus.

5. The method of claim 1, wherein:
separating the adduct comprises magnetically capturing the microparticle.

6. The method of claim 1, wherein:
separating the adduct comprises capturing the microparticle by filtration.

7. The method of claim 1, wherein:
separating the adduct comprises capturing the microparticle by centrifugation.

8. The method of claim 1, wherein:
the receptor comprises at least one binding protein.

9. The method of claim 1, wherein:
the receptor comprises at least one antibody.

10. The method of claim 9, wherein:
the biological sample is a forensic sample and the target species is a sperm cell.

11. The method of claim 10, wherein:
the separated component of the target species includes a DNA; the method further comprising:
magnetically removing the adduct; and
analyzing the DNA.

12. The method of claim 1, wherein:
the microparticle has a diameter in the range of from about 1 millimeter to 200 nanometers.

13. The method of claim 1, wherein:
the microparticle has a diameter in the range of from about 1 millimeter to about 500 nanometers.

14. The method of claim 1, wherein:
the microparticle has a diameter in the range of from about 1 millimeter to about 1 micrometer.

15. A separation reagent for a biological sample comprising:
a microparticle;
a receptor coupled to the microparticle; and
a photoaffinity label coupled to the receptor.

16. The separation reagent of claim 15, wherein:
the photoaffinity label is coupled to the receptor at an N-terminus.

17. The separation reagent of claim 15, wherein:
the microparticle includes a magnetic bead.

18. The separation reagent of claim 15, wherein:
the receptor comprises at least one binding protein.

19. The separation reagent of claim 15, wherein:
the receptor comprises at least one antibody.

20. The separation reagent of claim 15, wherein:
the photoaffinity label comprises an arylazide.

21. The separation reagent of claim 20, wherein:
the arylazide comprises a nitroarylazide.

22. The separation reagent of claim 15, wherein:
the photoaffinity label is sulfosuccinimidyl-perfluoroazidobenzamido-ethyl-1,3′-dithiopropionate, sulfosuccinimidyl-2-[m-azido-o-nitrobenzamido]ethyl-1,3′-dithiopropionate, N-succinimidyl-4-azidophenyl-1,3′-dithiopropionate or sulfosuccinimidyl 2-[7-azido-4-methyl-coumarin-3-acetamido]ethyl-1,3′-dithiopropionate.

23. The separation reagent of claim 15, wherein:
the microparticle has a diameter in the range of from about 1 millimeter to 200 nanometers.

24. The separation reagent of claim 15, wherein:
the microparticle has a diameter in the range of from about 1 millimeter to about 500 nanometers.

25. The separation reagent of claim 15, wherein:
the microparticle has a diameter in the range of from about 1 millimeter to about 1 micrometer.

26. An apparatus for separating components of a biological sample, comprising:
a first chamber for receiving a biological sample;
a first capture means proximate to the first chamber for capturing a separation reagent;
a second chamber in fluidic communication with the first chamber; and
a second capture means proximate to the second chamber for capturing the separation reagent.
